(19) United States
(12) Patent Application Publication
(10) Pub. No.: US 2006/0024196 A1
(43) Pub. Date: Feb. 2, 2006

(54) Antimicrobial and deodorizing product

(75) Inventor: Yasuhiro Shimada, Tokyo (JP)

Correspondence Address: Norman P. Soloway;HAYES SOLOWAY P.C., 130 W. Cushing Street, Tucson, AZ, 85701, US

(73) Assignee: DELPHI LABORATORIES, INC.

(21) Appl. No.: 11/152,863

(22) Filed: Jun. 15, 2005

(30) Foreign Application Priority Data
Jul. 30, 2004 (JP) 00040701

(51) Int. Cl. A61L 2/238

(52) U.S. Cl. 422/28; 424/76.9; 424/600

(57) ABSTRACT

An indoor anti-microbial and deodorizing coating solution comprising titanium oxide and titanium phosphate in a liquid carrier.

### CROSS REFERENCE TO RELATED APPLICATIONS

The present application claims priority under 35 USC § 119 from Japanese Patent Application Serial No. 00040701, filed Jul. 30, 2004, the contents of which are incorporated by reference.

### BACKGROUND OF THE INVENTION

1. Field of Invention

The present invention relates generally to antimicrobial and deodorizing products, and more particularly, to sterilizing and deodorizing products that are safe and nonpoisonous and have a lasting effect. The invention has particularly utility in treating surfaces in closed spaces such as building walls and will be described in connection with such utility, although other utilities such as for sterilizing and deodorizing closed vehicles such as cars and trains, planes and boats, clothing and shoes, etc., are contemplated.

2. Description of the Prior Art

Indoor air quality is increasingly an issue in the United States and elsewhere because improvements in construction have made buildings more air tight in order to achieve energy efficiency in heating and cooling. Without adequate ventilation, indoor air pollutants are more likely to build up and may be the source of health concerns. Typical pollutants include mold and bacteria including the Legionella bacterium, as well as various chemical outgases such as formaldehyde, acetaldehyde, toluene, styrene, ethyl benzene, zylene and other chemicals which may lead to so-called “sick house” syndrome. Odors including cooking odors, body odors, spoiled food odors, smoke, pet odors, bathroom odors, perspiration and the like may also build up in a closed environment.

Recently it has been proposed to use a photocatalyst such as titanium oxide TiOx (1<x<2) as an antibacterial/deodorizer to make toilets self-cleaning. Titanium oxide will generate an electron with strong reduction capabilities and an electron hole with strong oxidative capacity under UV radiation. Thus, organic compounds that come in contact with titanium oxide under UV radiation will decompose according to an oxidation/reduction reaction. More specifically, the titanium oxide acts as a catalyst whereby to decompose or deodorize organic materials that come in contact with it. Because the titanium oxide acts as a catalyst, it is not consumed. Thus, the titanium oxide will last for a long time.

However, titanium oxide has two drawbacks. First, it only works in the presence of strong light. Secondly, it is potentially corrosive or harmful to many surfaces (wood, metal and concrete). Thus, titanium oxide catalytic antibacterial/deodorizer has been employed primarily on ceramic and the like surfaces.

Titanium phosphate also has been used as a catalytic topical cleaning agent. Titanium phosphate like titanium dioxide acts as a catalyst in the decomposition of the organic materials and the like. However, unlike titanium oxide, titanium phosphate is activated by the presence of moisture and oxygen. Thus, titanium phosphate primarily has been used as a catalytic additive to cleaning materials.

### SUMMARY OF THE INVENTION

The present invention provides an advanced type of antibacterial and deodorizing material which combines a photocatalyst, such as titanium oxide, preferably titanium dioxide (TiO₂) and an oxygen/moisture activated catalyst such as titanium phosphate in an easy to apply solution which may be applied to surfaces such as walls and dried in place leaving both titanium oxide and titanium phosphate on the surface. The resultant treated surface will continuously break down volatile toxic organic substances and odors both in the presence of UV light or oxygen and moisture. Optionally, silver ions up to 5 to 10% by weight, and/or one or more additional bactericidal substances, e.g. essential oils or phytoncides obtained from various plants including eucalyptus, onion, rosemary and the like, up to 5 to 10% by weight, may be included in the solution.

The titanium phosphate in the mixture sets up before the titanium oxide and helps to create a protective coating protecting the surface to which the product is applied against potentially corrosive or harmful action of the titanium oxide on that surface as well as acting as part of the binder to that surface. Moreover, the mixture of titanium oxide and titanium phosphate appears to be synergistic when applied to surfaces in combination with silver ions and/or one or more phytoncides.

The solution of titanium oxide and titanium phosphate made in accordance with the present invention has been found to be effective in controlling and reducing bacteria, virus, mold and mildew. The solutions also are effective against odors such as cooking odors, body odors, spoiled food odors, smoke, pet odors, bathroom odors, perspiration, and tobacco and also various outgassed chemicals such as ethylbenzene, formaldehyde, acetaldehyde, toluene, styrene, and other volatiles, as well as nitric and sulfuric acids. The solution may be applied to wall, floor and ceiling surfaces, fabric, leather goods and the like, including clothing, shoes, linen, storage closets, etc., automobile, airplane, train and boat cabins leaving the treated surfaces and surrounding areas clean smelling and virus and mold free.

Preparation of the catalytic sterilizing and deodorizing product of the present invention will be described below.

Dry titanium oxide and titanium phosphate particles are ground to fine powder, typically between one millimicron and one micron average size and mixed in a carrier such as water. Grinding the particles small enough results in the particles essentially remaining suspended in solution in the water. If the particles are larger than colloidal dimensions they will tend to precipitate if heavier than the suspending medium, or to agglomerate and rise to the surface, if lighter. Also, the smaller the particles, the more effective the product per unit and the more transparent it is once applied and dried on a surface. However, if desired, the carrier may be stirred to assist in maintaining the titanium oxide and the titanium phosphate particles in suspension. If desired, one or more phytoncides, silver ions and/or metal oxides such as zinc oxide or tin oxide also may be added to the solution to enhance its deodorizing effects, bacterial resistance and anti-mildew effects. The suspended solution may then be applied to a surface, such as a wall, by brushing or spray coating, and dried in place.

The ratio of titanium oxide to titanium phosphate contained in the solution may vary widely. Typically, the ratio of titanium oxide to titanium phosphate may be from 0.1 to 10:10 to 0.1 parts by weight. Preferably the titanium oxide and titanium phosphate are present in equal amounts, by weight. The solids loading of the solution also may vary widely. However, from a standpoint of economy of storage and shipment, and ease of application, the solids loading should be as close to saturation as possible.

Further features and advantages of the present invention will be seen from the following working examples:

A suspension of equal parts by weight of titanium oxide and titanium phosphate in water is sprayed onto a test surface, and allowed to dry. The following tests were run:

Anti-Bacterial and Anti-Mold Effect

Anti-Bacterial Test

The present invention has been shown effective against klebsiella pneumoniae and staphylococcus aureus. In a laboratory test employing standard measurement techniques in which a nutrient broth was cultured for 18 hours at 37° C. for treated an untreated surfaces, both the bacteriostatic activity rate and the antibacterial activity rate exceeded established thresholds (of 2.2 and 0.0 respectively). Performing the same test on the present invention after having washed five times the cloth on to which the invention had been applied resulted in a live bacterial count of 7.6×10⁴ which resulted in a bacteriostatic activity rate of 2.8 and an antibacterial activity rate of 0.2. In contrast, the untreated surface yielded a substantially higher live bacterial count of 3.4×10⁷. Thus, even after five washings, the invention retained some of its antibacterial properties.

Anti-Mold Test

Several less formal tests have also been made on molds. Significant reduction in an unspecified mold in a sheet of plaster of paris was visually determined in 3 days. The same surface was essentially mold free two months later. A further test of black mold from grape juice on an outside surface showed significant reduction in 3 days. A further test of the invention against a combination of mold and bacteria on an outside surface showed a substantial difference between an outside wooden surface half of which was treated with the invention and half of which was not.

Deodorizing Effect

The ability of the present invention to remove offensive odors was tested in connection with three causes of odors—hydrogen sulfide, ammonia and formaldehyde. (Initial concentrations were 20 ppm, 50 ppm and 8 ppm respectively.) After placing 1.06 g of the invention on a glass plate (10 cm×10 cm) in a 2 liter container for six hours, the concentrations of the three gases were measured again. The resulting concentrations were 13 ppm, 1 ppm and 0 ppm respectively, resulting in reducing effectiveness of 35%, 98% and 100% against the three odors. Similar results have been measured against acetaldehyde—a major cause of tobacco smell. Other studies show that the impact of the invention upon target substances is not immediate, but, depending on the balance of the volume of the product and the concentration of the target substance, sometimes takes hours or days before the resulting concentrations fall below 1 ppm.

VOC Reduction

The present invention has also been demonstrated effective in reducing certain volatile organic compounds. Tests conducted in public high schools in Japan, for example yield the following results:

| | Formaldehyde (0.08)* | | | | Toluene (0.07)* | | | | Xylene (0.20)* | | | |
|---|---|---|---|---|---|---|---|---|---|---|---|---|
| Room | Prior | 1st | 2nd | 3rd | Prior | 1st | 2nd | 3rd | Prior | 1st | 2nd | 3rd |
| 1A | 0.088 | 0.040 | 0.005 | 0 | 0.355 | 0.080 | 0.060 | 0.020 | 0.710 | 0.160 | 0.120 | 0.040 |
| 1B | 0.088 | 0.044 | 0.005 | 0 | 0.250 | 0.100 | 0.080 | 0.020 | 0.500 | 0.200 | 0.160 | 0.040 |
| 1C | 0.110 | 0.052 | 0.005 | 0 | 0.357 | 0.130 | 0.100 | 0.035 | 0.750 | 0.260 | 0.200 | 0.070 |
| 2A | 0.105 | 0.024 | 0.010 | 0 | 0.280 | 0.070 | 0.050 | 0.020 | 0.560 | 0.140 | 0.100 | 0.040 |
| 2B | 0.100 | 0.030 | 0.010 | 0 | 0.265 | 0.130 | 0.100 | 0.025 | 0.530 | 0.260 | 0.200 | 0.050 |
| 2C | 0.105 | 0.020 | 0.010 | 0 | 0.300 | 0.130 | 0.100 | 0.030 | 0.600 | 0.260 | 0.200 | 0.060 |
| 3A | 0.100 | 0.060 | 0.007 | 0 | 0.400 | 0.100 | 0.080 | 0.030 | 0.800 | 0.200 | 0.160 | 0.060 |
| 3B | 0.105 | 0.050 | 0.007 | 0 | 0.350 | 0.120 | 0.100 | 0.050 | 0.700 | 0.240 | 0.200 | 0.100 |
| 3C | 0.100 | 0.040 | 0.009 | 0 | 0.350 | 0.120 | 0.100 | 0.050 | 0.700 | 0.240 | 0.200 | 0.100 |

*bracketed amounts are Japanese Ministry of Health Guidelines Units: ppm

Measurement dates and times

Prior to application

| | |
|---|---|
| 1st test after applicant | 5 days later |
| 2nd test after application | 14 days later |
| 3rd test after application | 21 days later |

Locations where measurements were taken:

| | | | |
|---|---|---|---|
| 1st Floor | 1A: large room | 1B: elective | 1C: dressing room |
| | | classroom | |
| 2nd Floor | 2A: boys room | 2B: classroom | 2C: classroom |
| 3rd Floor | 3A: hallway/lobby | 3B: classroom | 3C: classroom |

The present invention has several features and advantages over the prior art. For one, the titanium phosphate acts as a protective binder against the corrosive effects of the titanium dioxide on the surface to which the product is applied. That is because the titanium phosphate sets up before the titanium dioxide and helps to create a protective binder for the surface to which the product is applied against the potentially corrosive or destructive power of the titanium dioxide itself on many surfaces to which it is applied. This feature makes it much easier to apply the titanium dioxide, enabling it to be applied outside of a factory setting. Also, phytoncides may be added to provide an essentially immediate impact upon odors when applied. The present invention, however, demonstrates a greater immediate effect than if only phytoncides had been applied. This result is unexpected because both titanium dioxide and titanium phosphate were thought take time to have an affect on volatile organic compounds as well as mold, bacteria and fungus. One possible explanation for the observed synergistic effect is that the phytoncides are broken up into smaller particles in the spray of the present invention because of the small size of the titanium dioxide, titanium phosphate or other ingredients.

Various changes may be made in the foregoing without departing from the spirit and scope of the invention. For example, other carriers which may be water based or oil based may be used. However, water is preferred in that it will not add to the volatile load in the building. Also, the titanium dioxide/titanium phosphate mixture may be added to paint and applied to wall surfaces, etc., or the titanium oxide and titanium phosphate may be added as an aerosol.

This is thus seen the present invention provides a coating exhibiting anti bacterial and deodorizing activities that are long lasting and effective against a variety of organic materials.

1. An indoor anti-microbial and deodorizing coating solution comprising titanium oxide and titanium phosphate particles in a liquid carrier.

2. The coating solution of claim 1, wherein the titanium oxide comprises titanium dioxide.

3. The indoor coating solution as claimed in claim 1, wherein the titanium oxide and titanium phosphate particles are between 1 millimicron and 1 micron average size.

4. The coating solution as claimed in claim 1, wherein the carrier comprises an aqueous based carrier.

5. The coating solution as claimed in claim 4, wherein the aqueous solution comprises water.

6. The coating solution as claimed in claim 1, further comprising one or more phytoncides.

7. The coating solution as claimed in claim 6, wherein the phytoncide comprises 5 to 10% by weight of the solution.

8. The coating solution as claimed in claim 1, further comprising silver ions.

9. The coating solution as claimed in claim 8, wherein the silver ions comprise up to 5 to 10% by weight of the solution.

10. The coating solution as claimed in claim 1, further comprising zinc oxide or tin oxide.

11. The coating solution as claimed in claim 1, wherein the titanium oxide and titanium phosphate are present in a ratio of 0.1 to 10:10 to 0.1 parts by weight.

12. The coating solution as claimed in claim 11, wherein the titanium oxide and titanium phosphate are present in equal amounts.

13. The coating solution as claimed in claim 11, wherein the titanium oxide and titanium phosphate are applied in an aerosol.

14. A method of sterilizing and deodorizing a surface which comprises applying the coating solution of claim 1 to the surface and drying the solution in place.
